(19) United States
(12) Patent Application Publication
(10) Pub. No.: US 2024/0277435 A1
(43) Pub. Date: Aug. 22, 2024

(54) ASYMMETRIC DRIVE INPUT LAYOUT WITH EQUIVALENT CABLE COMPLIANCE

(71) Applicant: Cilag GmbH International, Zug (CH)

(72) Inventor: Austin Michael FISCHER, Cincinnati, OH (US)

(73) Assignee: Cilag GmbH International, Zug (CH)

(21) Appl. No.: 18/170,238

(22) Filed: Feb. 16, 2023

(51) Int. Cl. A61B 34/00; A61B 34/37

(52) U.S. Cl. CPC A61B 34/71; A61B 34/37; A61B 2034/302

(57) ABSTRACT

A surgical tool includes a drive housing having opposing distal and proximal ends, first and second drive cable capstans arranged within the drive housing and asymmetrically aligned between the distal and proximal ends, a first drive cable extending distally from the drive housing and actuatable by operation of the first drive cable capstan, and a second drive cable extending distally from the drive housing and actuatable by operation of the second drive cable capstan. A stiffness of the first and second drive cables is equalized by adjusting one or more characteristics of one or both of the first and second drive cables.

### BACKGROUND

Minimally invasive surgical (MIS) instruments are often preferred over traditional open surgical devices due to reduced post-operative recovery time and minimal scarring. Laparoscopic surgery is one type of MIS procedure in which one or more small incisions are formed in the abdomen of a patient and a trocar is inserted through the incision to form a pathway that provides access to the abdominal cavity. Through the trocar, a variety of instruments and surgical tools can be introduced into the abdominal cavity. The instruments and tools introduced into the abdominal cavity via the trocar can be used to engage and/or treat tissue in a number of ways to achieve a diagnostic or therapeutic effect.

Various robotic systems have been developed to assist in MIS procedures. Robotic systems can allow for more instinctive hand movements by maintaining natural eye-hand axis. Robotic systems can also allow for more degrees of freedom in movement by including an articulable “wrist” joint that creates a more natural hand-like articulation. In such systems, an end effector positioned at the distal end of the instrument can be articulated (moved) using a cable driven motion system having one or more drive cables that extend through the wrist joint. A user (e.g., a surgeon) is able to remotely operate the end effector by grasping and manipulating in space one or more controllers that communicate with a tool driver coupled to the surgical instrument. User inputs are processed by a computer system incorporated into the robotic surgical system, and the tool driver responds by actuating the cable driven motion system. Moving the drive cables articulates the end effector to desired angular positions and configurations.

Some cable driven motion systems utilize antagonistic cable designs with multiple drive inputs to drive end effector functionality and articulation. Some of the drive cables exhibit different cable pathway lengths, which can result in different compliances for each drive input that drives actuation of the drive cable. This can result in controls complications.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an example robotic surgical system that may incorporate some or all of the principles of the present disclosure.

FIG. 2 is an isometric side view of an example surgical tool that may incorporate some or all of the principles of the present disclosure.

FIG. 3 illustrates potential degrees of freedom in which the wrist of the surgical tool of FIG. 2 may be able to articulate (pivot) and translate.

FIG. 4 is an enlarged isometric view of the distal end of the surgical tool of FIG. 2.

FIG. 5 is a bottom view of the drive housing of FIG. 2, according to one or more embodiments.

FIGS. 6A and 6B are exposed, isometric right and left views, respectively, of the interior of the drive housing of FIG. 2.

FIG. 7 is a top, exposed view of the drive housing of FIG. 2, according to one or more embodiments.

FIG. 8 is a schematic view of the drive cables as arranged within the drive housing of FIG. 2.

FIG. 9 is a schematic side view of an example drive cable, according to one or more embodiments.

### DETAILED DESCRIPTION

The present disclosure is related to robotic surgical systems and, more particularly, to optimizing characteristics of surgical tool drive cables to create equivalent compliance between drive inputs that actuate the drive cables.

Embodiments discussed herein describe a cable driven drive system having an asymmetric layout of drive inputs that control operation of end effector jaws and articulation. A drive member or cable is operatively connected to each of the drive inputs and is composed of multiple sections having separate stiffnesses. The separate stiffnesses is achieved by using different materials, cross-sections, braid constructions, and other characteristics of the drive cables. The drive cables described herein can have one or more flexible regions or cables made of, for example, tungsten, a stainless steel braid, or a polymer fiber, and coupled to a secondary stiffness component. One or more characteristics of the drive cables can be adjusted and thereby used as a compliance corrector, which can be designed such that the stiffness of one cable pathway is the same as another cable pathway of separate length. In some applications, for example, the length of the stiffness component can be increased to increase the overall stiffness of the drive cable to be equivalent to that of a shorter pathway for another drive cable.

The embodiments described herein may be advantageous in creating equivalent compliance between each drive input, which can simplify the robotic control scheme and increase performance accuracy significantly. This may also enable asymmetric layout of the drive inputs, which provides more flexibility in design for other device functionality.

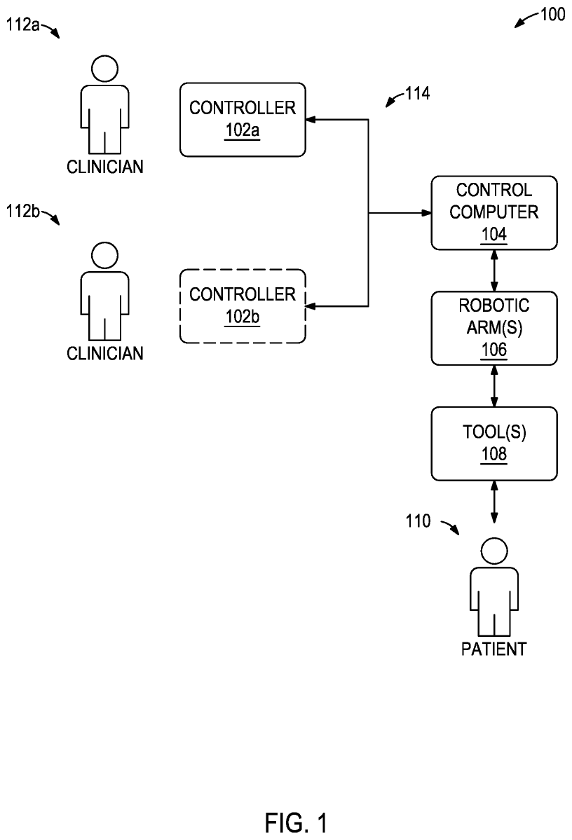

FIG. 1 is a block diagram of an example robotic surgical system 100 that may incorporate some or all of the principles of the present disclosure. As illustrated, the system 100 can include at least one set of user input controllers 102a and at least one control computer 104. The control computer 104 may be mechanically and/or electrically coupled to a robotic manipulator and, more particularly, to one or more robotic arms 106 (alternately referred to as “tool drivers”). In some embodiments, the robotic manipulator may be included in or otherwise mounted to an arm cart capable of making the system portable. Each robotic arm 106 may include and otherwise provide a location for mounting one or more surgical instruments or tools 108 for performing various surgical tasks on a patient 110. Operation of the robotic arms 106 and associated tools 108 may be directed by a clinician 112a (e.g., a surgeon) from the user input controller 102a.

In some embodiments, a second set of user input controllers 102b (shown in dashed line) may be operated by a second clinician 112b to direct operation of the robotic arms 106 and tools 108 via the control computer 104 and in conjunction with the first clinician 112a. In such embodiments, for example, each clinician 112a,b may control different robotic arms 106 or, in some cases, complete control of the robotic arms 106 may be passed between the clinicians 112a,b as needed. In some embodiments, additional robotic manipulators having additional robotic arms may be utilized during surgery on the patient 110, and these additional robotic arms may be controlled by one or more of the user input controllers 102a,b.

The control computer 104 and the user input controllers 102a,b may be in communication with one another via a communications link 114, which may be any type of wired or wireless telecommunications means configured to carry a variety of communication signals (e.g., electrical, optical, infrared, etc.) according to any communications protocol. In some applications, for example, there is a tower with ancillary equipment and processing cores designed to drive the robotic arms 106.

The user input controllers 102a,b generally include one or more physical controllers that can be grasped by the clinicians 112a,b and manipulated in space while the surgeon views the procedure via a stereo display. The physical controllers generally comprise manual input devices movable in multiple degrees of freedom, and which often include an actuatable handle for actuating the surgical tool(s) 108, for example, for opening and closing opposing jaws, applying an electrical potential (current) to an electrode, or the like. The control computer 104 can also include an optional feedback meter viewable by the clinicians 112a,b via a display to provide a visual indication of various surgical instrument metrics, such as the amount of force being applied to the surgical instrument (i.e., a cutting instrument or dynamic clamping member).

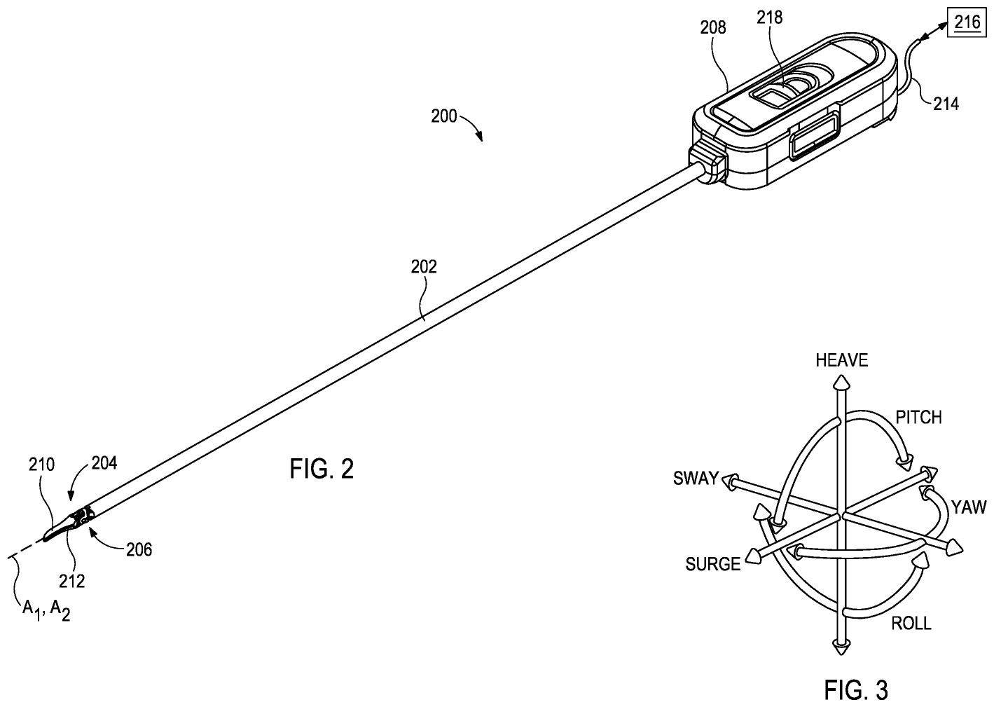

FIG. 2 is an isometric side view of an example surgical tool 200 that may incorporate some or all of the principles of the present disclosure. The surgical tool 200 may be the same as or similar to the surgical tool(s) 108 of FIG. 1 and, therefore, may be used in conjunction with a robotic surgical system, such as the robotic surgical system 100 of FIG. 1. Accordingly, the surgical tool 200 may be designed to be releasably coupled to a tool driver included in the robotic surgical system 100. In other embodiments, however, aspects of the surgical tool 200 may be adapted for use in a manual or hand-operated manner, without departing from the scope of the disclosure.

As illustrated, the surgical tool 200 includes an elongated shaft 202, an end effector 204, a wrist 206 (alternately referred to as a “wrist joint” or an “articulable wrist joint”) that couples the end effector 204 to the distal end of the shaft 202, and a drive housing 208 coupled to the proximal end of the shaft 202. In applications where the surgical tool is used in conjunction with a robotic surgical system (e.g., the robotic surgical system 100 of FIG. 1), the drive housing 208 can include coupling features that releasably couple the surgical tool 200 to the robotic surgical system.

The terms “proximal” and “distal” are defined herein relative to a robotic surgical system having an interface configured to mechanically and electrically couple the surgical tool 200 (e.g., the housing 208) to a robotic manipulator. The term “proximal” refers to the position of an element closer to the robotic manipulator and the term “distal” refers to the position of an element closer to the end effector 204 and thus further away from the robotic manipulator. Alternatively, in manual or hand-operated applications, the terms “proximal” and “distal” are defined herein relative to a user, such as a surgeon or clinician. The term “proximal” refers to the position of an element closer to the user and the term “distal” refers to the position of an element closer to the end effector 204 and thus further away from the user. Moreover, the use of directional terms such as above, below, upper, lower, upward, downward, left, right, and the like are used in relation to the illustrative embodiments as they are depicted in the figures, the upward or upper direction being toward the top of the corresponding figure and the downward or lower direction being toward the bottom of the corresponding figure.

During use of the surgical tool 200, the end effector 204 is configured to move (pivot) relative to the shaft 202 at the wrist 206 to position the end effector 204 at desired orientations and locations relative to a surgical site. To accomplish this, the housing 208 includes (contains) various drive inputs and mechanisms (e.g., gears, actuators, etc.) designed to control operation of various features associated with the end effector 204 (e.g., clamping, firing, cutting, rotation, articulation, etc.). In at least some embodiments, the shaft 202, and hence the end effector 204 coupled thereto, is configured to rotate about a longitudinal axis A₁ of the shaft 202. In such embodiments, at least one of the drive inputs included in the housing 208 is configured to control rotational movement of the shaft 202 about the longitudinal axis A₁.

The shaft 202 is an elongate member extending distally from the housing 208 and has at least one lumen extending therethrough along its axial length. In some embodiments, the shaft 202 may be fixed to the housing 208, but could alternatively be rotatably mounted to the housing 208 to allow the shaft 202 to rotate about the longitudinal axis A₁. In yet other embodiments, the shaft 202 may be releasably coupled to the housing 208, which may allow a single housing 208 to be adaptable to various shafts having different end effectors.

The end effector 204 can exhibit a variety of sizes, shapes, and configurations. In the illustrated embodiment, the end effector 204 comprises a combination tissue grasper and vessel sealer that include opposing first (upper) and second (lower) jaws 210, 212 configured to move (articulate) between open and closed positions. As will be appreciated, however, the opposing jaws 210, 212 may alternatively form part of other types of end effectors such as, but not limited to, a surgical scissors, a clip applier, a needle driver, a babcock including a pair of opposed grasping jaws, bipolar jaws (e.g., bipolar Maryland grasper, forceps, a fenestrated grasper, etc.), etc. One or both of the jaws 210, 212 may be configured to pivot to articulate the end effector 204 between the open and closed positions.

FIG. 3 illustrates the potential degrees of freedom in which the wrist 206 may be able to articulate (pivot) and thereby move the end effector 204. The wrist 206 can have any of a variety of configurations. In general, the wrist 206 comprises a joint configured to allow pivoting movement of the end effector 204 relative to the shaft 202. The degrees of freedom of the wrist 206 are represented by three translational variables (i.e., surge, heave, and sway), and by three rotational variables (i.e., Euler angles or roll, pitch, and yaw). The translational and rotational variables describe the position and orientation of the end effector 204 with respect to a given reference Cartesian frame. As depicted in FIG. 3, “surge” refers to forward and backward translational movement, “heave” refers to translational movement up and down, and “sway” refers to translational movement left and right. With regard to the rotational terms, “roll” refers to tilting side to side, “pitch” refers to tilting forward and backward, and “yaw” refers to turning left and right.

The pivoting motion can include pitch movement about a first axis of the wrist 206 (e.g., X-axis), yaw movement about a second axis of the wrist 206 (e.g., Y-axis), and combinations thereof to allow for 360° rotational movement of the end effector 204 about the wrist 206. In other applications, the pivoting motion can be limited to movement in a single plane, e.g., only pitch movement about the first axis of the wrist 206 or only yaw movement about the second axis of the wrist 206, such that the end effector 204 moves only in a single plane.

Referring again to FIG. 2, the surgical tool 200 may also include a plurality of drive cables (obscured in FIG. 2) that form part of a cable driven motion system configured to facilitate actuation and articulation of the end effector 204 relative to the shaft 202. Moving (actuating) one or more of the drive cables moves the end effector 204 between an unarticulated position and an articulated position. The end effector 204 is depicted in FIG. 2 in the unarticulated position where a longitudinal axis A₂ of the end effector 204 is substantially aligned with the longitudinal axis A₁ of the shaft 202, such that the end effector 204 is at a substantially zero angle relative to the shaft 202. Due to factors such as manufacturing tolerance and precision of measurement devices, the end effector 204 may not be at a precise zero angle relative to the shaft 202 in the unarticulated position, but nevertheless be considered “substantially aligned” thereto. In the articulated position, the longitudinal axes A₁, A₂ would be angularly offset from each other such that the end effector 204 is at a non-zero angle relative to the shaft 202.

In some embodiments, the surgical tool 200 may be supplied with electrical power (current) via a power cable 214 coupled to the housing 208. In other embodiments, the power cable 214 may be omitted and electrical power may be supplied to the surgical tool 200 via an internal power source, such as one or more batteries, capacitors, or fuel cells. In such embodiments, the surgical tool 200 may alternatively be characterized and otherwise referred to as an “electrosurgical instrument” capable of providing electrical energy to the end effector 204.

The power cable 214 may place the surgical tool 200 in electrical communication with a generator 216 that supplies energy, such as electrical energy (e.g., radio frequency energy), ultrasonic energy, microwave energy, heat energy, or any combination thereof, to the surgical tool 200 and, more particularly, to the end effector 204. Accordingly, the generator 216 may comprise a radio frequency (RF) source, an ultrasonic source, a direct current source, and/or any other suitable type of electrical energy source that may be activated independently or simultaneously.

In applications where the surgical tool 200 is configured for bipolar operation, the power cable 214 will include a supply conductor and a return conductor. Current can be supplied from the generator 216 to an active (or source) electrode located at the end effector 204 via the supply conductor, and current can flow back to the generator 216 via a return electrode located at the end effector 204 via the return conductor. In the case of a bipolar grasper with opposing jaws, for example, the jaws serve as the electrodes where the proximal end of the jaws are isolated from one another and the inner surface of the jaws (i.e., the area of the jaws that grasp tissue) apply the current in a controlled path through the tissue. In applications where the surgical tool 200 is configured for monopolar operation, the generator 216 transmits current through a supply conductor to an active electrode located at the end effector 204, and current is returned (dissipated) through a return electrode (e.g., a grounding pad) separately coupled to a patient's body.

The surgical tool 200 may further include a manual release switch 218 that may be manually actuated by a user (e.g., a surgeon) to open the jaws 210, 212. The release switch 218 is movably positioned on the drive housing 208, and a user is able to manually move (slide) the release switch 218 from a disengaged position, as shown, to an engaged position. In the disengaged position, the surgical tool 200 is able to operate as normal. As the release switch 218 moves to the engaged position, however, various internal component parts of the drive housing 208 are simultaneously moved, thereby resulting in the jaws 210, 212 opening, which might prove beneficial for a variety of reasons. In some applications, for example, the release switch 218 may be moved in the event of an electrical disruption that renders the surgical tool 200 inoperable. In such applications, the user would be able to manually open the jaws 210, 212 and thereby release any grasped tissue and remove the surgical tool 200. In other applications, the release switch 218 may be actuated (enabled) to open the jaws 210, 212 in preparation for cleaning and/or sterilization of the surgical tool 200. In some applications, the surgical tool 200 is first decoupled from the robotic manipulator and the associated motors, following which the user can actuate the manual release switch 218 to move the associated inputs and drive the cables once the motors are disengaged.

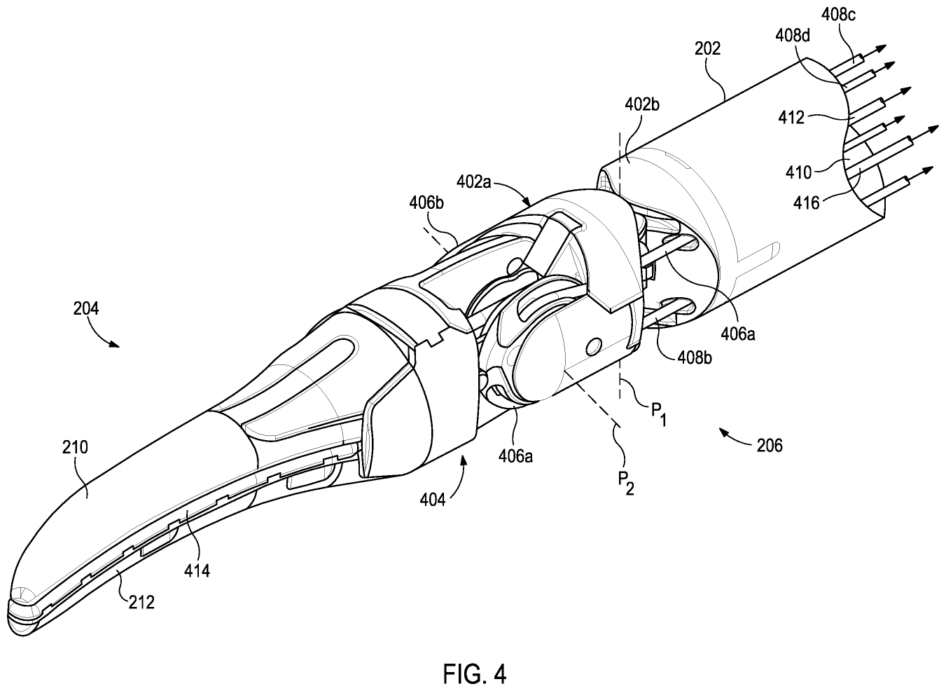

FIG. 4 is an enlarged isometric view of the distal end of the surgical tool 200. More specifically, FIG. 4 depicts an enlarged view of the end effector 204 and the wrist 206, with the jaws 210, 212 of the end effector 204 in the closed position. The wrist 206 operatively couples the end effector 204 to the shaft 202. In some embodiments, however, a shaft adapter may be directly coupled to the wrist 206 and otherwise interpose the shaft 202 and the wrist 206. Accordingly, the wrist 206 may be operatively coupled to the shaft 202 either through a direct coupling engagement where the wrist 206 is directly coupled to the distal end of the shaft 202, or an indirect coupling engagement where a shaft adapter interposes the wrist 206 and the distal end of the shaft 202. As used herein, the term “operatively couple” refers to a direct or indirect coupling engagement between two components.

To operatively couple the end effector 204 to the shaft 202, the wrist 206 includes a first or “distal” clevis 402a and a second or “proximal” clevis 402b. The clevises 402a,b are alternatively referred to as “articulation joints” of the wrist 206 and extend from the shaft 202, or alternatively a shaft adapter. The clevises 402a,b are operatively coupled to facilitate articulation of the wrist 206 relative to the shaft 202. As illustrated, the wrist 206 also includes a linkage 404 arranged distal to the distal clevis 402a and operatively mounted to the jaws 210, 212.

As illustrated, the proximal end of the distal clevis 402a may be rotatably mounted or pivotably coupled to the proximal clevis 402b at a first pivot axis P₁ of the wrist 206. In some embodiments, an axle may extend through the first pivot axis P₁ and the distal and proximal clevises 402a,b may be rotatably coupled via the axle. In other embodiments, however, such as is depicted in FIG. 4, the distal and proximal clevises 402a,b may be engaged in rolling contact, such as via an intermeshed gear relationship that allows the clevises 402a,b to rotate relative to each other similar to a rolling joint.

First and second pulleys 406a and 406b may be rotatably mounted to the distal end of the distal clevis 402a at a second pivot axis P₂ of the wrist 206. The linkage 404 may be arranged distal to the second pivot axis P₂ and operatively mounted to the jaws 210, 212. The first pivot axis P₁ is substantially perpendicular (orthogonal) to the longitudinal axis A₁ of the shaft 202, and the second pivot axis P₂ is substantially perpendicular (orthogonal) to both the longitudinal axis A₁ and the first pivot axis P₁. Movement of the end effector 204 about the first pivot axis P₁ provides “yaw” articulation of the wrist 206, and movement about the second pivot axis P₂ provides “pitch” articulation of the wrist 206.

A plurality of drive cables, shown as drive cables 408a, 408b, 408c, and 408d, extend longitudinally within a lumen 410 defined by the shaft 202 (or a shaft adaptor) and extend at least partially through the wrist 206. The drive cables 408a-d may form part of the cable driven motion system housed within the drive housing 208 (FIG. 2), and may comprise cables, bands, lines, cords, wires, woven wires, ropes, strings, twisted strings, elongate members, belts, shafts, flexible shafts, drive rods, or any combination thereof. The drive cables 408a-d can be made from a variety of materials including, but not limited to, a metal (e.g., tungsten, stainless steel, nitinol, etc.), a polymer (e.g., ultra-high molecular weight polyethylene), a synthetic fiber (e.g., KEVLAR®, VECTRAN®, etc.), an elastomer, or any combination thereof. While four drive cables 408a-d are depicted in FIG. 4, more or less than four may be employed, without departing from the scope of the disclosure.

The drive cables 408a-d extend proximally from the end effector 204 and the wrist 206 toward the drive housing 208 (FIG. 2) where they are operatively coupled to various actuation mechanisms or devices that facilitate longitudinal movement (translation) of the drive cables 408a-d within the lumen 410. Selective actuation of the drive cables 408a-d applies tension (i.e., pull force) to the given drive cable 408a-d in the proximal direction, which urges the given drive cable 408a-d to translate longitudinally within the lumen 410.

In the illustrated embodiment, the drive cables 408a-d each extend longitudinally through the proximal clevis 402b. The distal end of each drive cable 408a-d terminates at the first or second pulleys 406a,b, thus operatively coupling each drive cable 408a-d to the end effector 204. In some embodiments, the distal ends of the first and second drive cables 408a,b may be coupled to each other and terminate at the first pulley 406a, and the distal ends of the third and fourth drive cables 408c,d may be coupled to each other and terminate at the second pulley 406b. In at least one embodiment, the distal ends of the first and second drive cables 408a,b and the distal ends of the third and fourth drive cables 408c,d may each be coupled together at corresponding ball crimps (not shown) mounted to the first and second pulleys 406a,b, respectively.

In at least one embodiment, the drive cables 408a-d may operate “antagonistically”. More specifically, when the first drive cable 408a is actuated (moved), the second drive cable 408b naturally follows as coupled to the first drive cable 408a, and when the third drive cable 408c is actuated, the fourth drive cable 408d naturally follows as coupled to the third drive cable 408c, and vice versa. Antagonistic operation of the drive cables 408a-d can open or close the jaws 210, 212 and can further cause the end effector 204 to articulate at the wrist 206. More specifically, selective actuation of the drive cables 408a-d in known configurations or coordination can cause the end effector 204 to articulate about one or both of the pivot axes P₁, P₂, thus facilitating articulation of the end effector 204 in both pitch and yaw directions. Moreover, selective actuation of the drive cables 408a-d in other known configurations or coordination will cause the jaws 210, 212 to open or close. Antagonistic operation of the drive cables 408a-d advantageously reduces the number of cables required to provide full wrist 206 motion, and also helps eliminate slack in the drive cables 408a-d, which results in more precise motion of the end effector 204.

In the illustrated embodiment, the end effector 204 is able to articulate (move) in pitch about the second or “pitch” pivot axis P₂, which is located near the distal end of the wrist 206. Thus, the jaws 210, 212 open and close in the direction of pitch. In other embodiments, however, the wrist 206 may alternatively be configured such that the second pivot axis P₂ facilitates yaw articulation of the jaws 210, 212, without departing from the scope of the disclosure.

In some embodiments, an electrical conductor 412 may also extend longitudinally within the lumen 410, through the wrist 206, and terminate at an electrode 414 to supply electrical energy to the end effector 204. In some embodiments, the electrical conductor 412 may comprise a wire, but may alternatively comprise a rigid or semi-rigid shaft, rod, or strip (ribbon) made of a conductive material. The electrical conductor 412 may be entirely or partially covered with an insulative covering (overmold) made of a non-conductive material. Using the electrical conductor 412 and the electrode 414, the end effector 204 may be configured for monopolar or bipolar RF operation.

In the illustrated embodiment, the end effector 204 comprises a combination tissue grasper and vessel sealer that includes a knife (not shown), alternately referred to as a “cutting element” or “blade.” The knife is aligned with and configured to traverse a guide track (not shown) defined longitudinally in one or both of the upper and lower jaws 210, 212. The knife may be operatively coupled to the distal end of a drive rod 416 that extends longitudinally within the lumen 410 and passes through the wrist 206. Longitudinal movement (translation) of the drive rod 416 correspondingly moves the knife within the guide track(s). Similar to the drive cables 408a-d, the drive rod 416 may form part of the actuation systems housed within the drive housing 208 (FIG. 2). Selective actuation of a corresponding drive input will cause the drive rod 416 to move distally or proximally within the lumen 410, and correspondingly move the knife 416 in the same longitudinal direction.

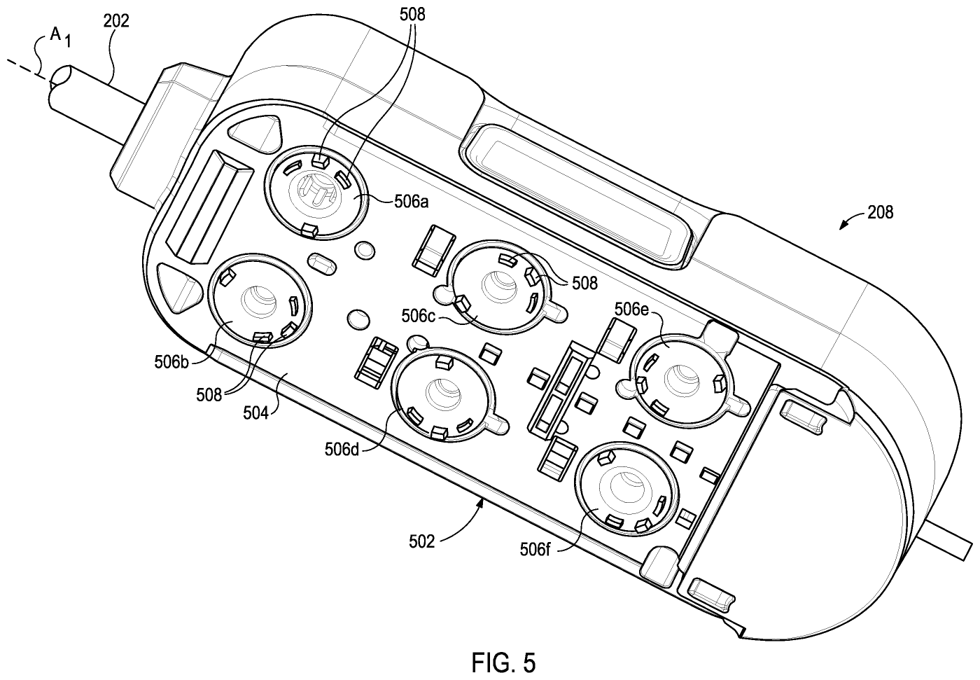

FIG. 5 is a bottom view of the drive housing 208, according to one or more embodiments. As illustrated, the drive housing 208 may include a tool mounting portion 502 used to operatively couple the drive housing 208 to a tool driver of a robotic manipulator. The tool mounting portion 502 may releasably couple the drive housing 208 to a tool driver in a variety of ways, such as by clamping thereto, clipping thereto, or slidably mating therewith. In some embodiments, the tool mounting portion 502 may include an array of electrical connecting pins, which may be coupled to an electrical connection on the mounting surface of the tool driver. While the tool mounting portion 502 is described herein with reference to mechanical, electrical, and magnetic coupling elements, it should be understood that a wide variety of telemetry modalities might be used, including infrared, inductive coupling, or the like.

The tool mounting portion 502 includes and otherwise provides an interface 504 configured to mechanically, magnetically, and/or electrically couple the drive housing 208 to the tool driver. As illustrated, the interface 504 includes and supports a plurality of inputs, shown as inputs 506a, 506b, 506c, 506d, 506e, and 506f. Each input 506a-f comprises a rotatable disc configured to align with and couple to a corresponding actuator of a given tool driver. Moreover, each input 506a-f provides or defines one or more surface features 508 configured to align with mating surface features provided on the corresponding actuator or drive output. The surface features 508 can include, for example, various protrusions and/or indentations that facilitate a mating engagement. In some embodiments, some or all of the inputs 506a-f may include one surface feature 508 that is positioned closer to an axis of rotation of the associated input 506a-f than the other surface feature(s) 508. This may help to ensure positive angular alignment of each input 506a-f.

In some embodiments, actuation of the first input 506a may be configured to control rotation of the elongate shaft 202 about its longitudinal axis A₁. The elongate shaft 202 may be rotated clockwise or counter-clockwise depending on the rotational actuation of the first input 506a. In some embodiments, actuation of the second, third, fourth, and fifth inputs 506b-e may be configured to operate movement (axial translation) of the drive cables 408a-d (FIG. 4), which results in the actuation of the wrist 106 (FIG. 4) and articulation (operation) of the end effector 204 (FIG. 4). In some embodiments, actuation of the sixth input 506f may be configured to advance and retract the drive rod 416 (FIG. 4), and thereby correspondingly advance or retract the knife at the end effector 204. Each of the inputs 506a-f may be actuated based on user inputs communicated to a tool driver coupled to the interface 504, and the user inputs may be received via a computer system incorporated into the robotic surgical system.

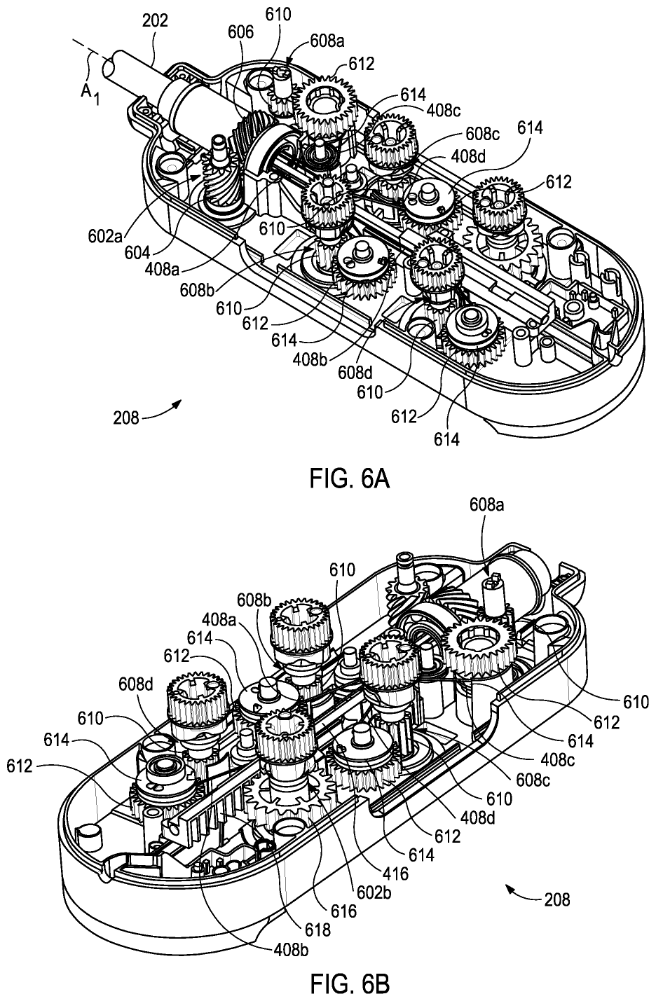

FIGS. 6A and 6B are exposed, isometric right and left views, respectively, of the interior of the drive housing 208, according to one or more embodiments. Several component parts that may be otherwise contained within the drive housing 208 are not shown in FIGS. 6A-6B to enable discussion of the depicted component parts. As best seen in FIG. 6A, a first capstan 602a is contained (housed) within the drive housing 208. The first capstan 602a may be operatively coupled to or extend from the first input 506a (FIG. 5) such that actuation of the first input 506a results in rotation of the first capstan 602a. A worm gear 604 is coupled to or forms part of the first capstan 602a. The worm gear 604 may be configured to mesh and interact with a driven gear 606 secured within the drive housing 208 and operatively coupled to the shaft 202 such that rotation of the driven gear 606 correspondingly rotates the shaft 202. Accordingly, rotation of the worm gear 604 (via actuation of the first input 506a of FIG. 5) will drive the driven gear 606 and thereby control rotation of the elongate shaft 202 about the longitudinal axis A₁.

The drive housing 208 may further contain or house a second capstan 602b operatively coupled to or extending from the sixth input 506f (FIG. 5) such that actuation of the sixth input 506f results in rotation of the second capstan 602b. As best seen in FIG. 6B, a drive gear 616 is coupled to or forms part of the second capstan 602b and is arranged to intermesh with a rack gear 618 positioned within the drive housing 208. The rack gear 618 is longitudinally translatable within the drive housing 208 as acted upon by the drive gear 616. The drive rod 416 may be operatively coupled to the rack gear 618 and extend distally therefrom to the end effector 204 (FIGS. 2 and 4). Accordingly, rotation of the second capstan 602b (via actuation of the sixth input 506f) will cause the rack gear 618 to longitudinally translate and correspondingly advance or retract the drive rod 416 and the knife coupled to the end of the drive rod 416 at the end effector 204.

The drive housing 208 further contains or houses a first drive cable capstan 608a, a second drive cable capstan 608b, a third drive cable capstan 608c, and a fourth drive cable capstan 608d. While four drive cable capstans 608a-d are depicted in FIGS. 6A-6B, alternative embodiments may include more or less than four, without departing from the scope of the disclosure. In the illustrated embodiment, the first drive cable capstan 608a is operatively coupled to or extends from the second input 506b (FIG. 5), the second drive cable capstan 608b is operatively coupled to or extends from the third input 506c (FIG. 5), the third drive cable capstan 608c is operatively coupled to or extends from the fourth input 506d (FIG. 5), and the fourth drive cable capstan 608d is operatively coupled to or extends from the fifth input 506e (FIG. 5). Accordingly, actuation of the second input 506b results in rotation of the first drive cable capstan 608a, actuation of the third input 506c results in rotation of the second drive cable capstan 608b, actuation of the fourth input 506d results in rotation of the third drive cable capstan 608c, and actuation of the fifth input 506e results in rotation of the fourth drive cable capstan 608d.

As illustrated, a corresponding drive gear 610 is coupled to or forms part of each drive cable capstan 608a-d. Moreover, each drive gear 610 is positioned to mesh and interact with a corresponding driven gear 612 rotatably mounted within the drive housing 208. Each driven gear 612 includes or is otherwise coupled to a corresponding cable pulley 614, and each cable pulley 614 is configured to be operatively coupled to (e.g., has wrapped there around) a corresponding one of the drive cables 408a-d. In the illustrated embodiment, the first drive cable 408a terminates at the cable pulley 614 ultimately driven by actuation of the second drive cable capstan 608b, the second drive cable 408b terminates at the cable pulley 614 ultimately driven by actuation of the fourth drive cable capstan 608d, the third drive cable 408c terminates at the cable pulley 614 ultimately driven by actuation of the first drive cable capstan 608a, and the fourth drive cable 408d terminates at the cable pulley 614 ultimately driven by actuation of the third drive cable capstan 608c.

Accordingly, rotation of the second drive cable capstan 608b (via actuation of the third input 506c of FIG. 5) will correspondingly control movement of the first drive cable 408a; rotation of the fourth drive cable capstan 608d (via actuation of the fifth input 506e of FIG. 5) will correspondingly control movement of the second drive cable 408b; rotation of the first drive cable capstan 608a (via actuation of the second input 506b of FIG. 5) will correspondingly control movement of the third drive cable 408c; and rotation of the third drive cable capstan 608c (via actuation of the fourth input 506d of FIG. 5) will correspondingly control movement of the fourth drive cable 408d.

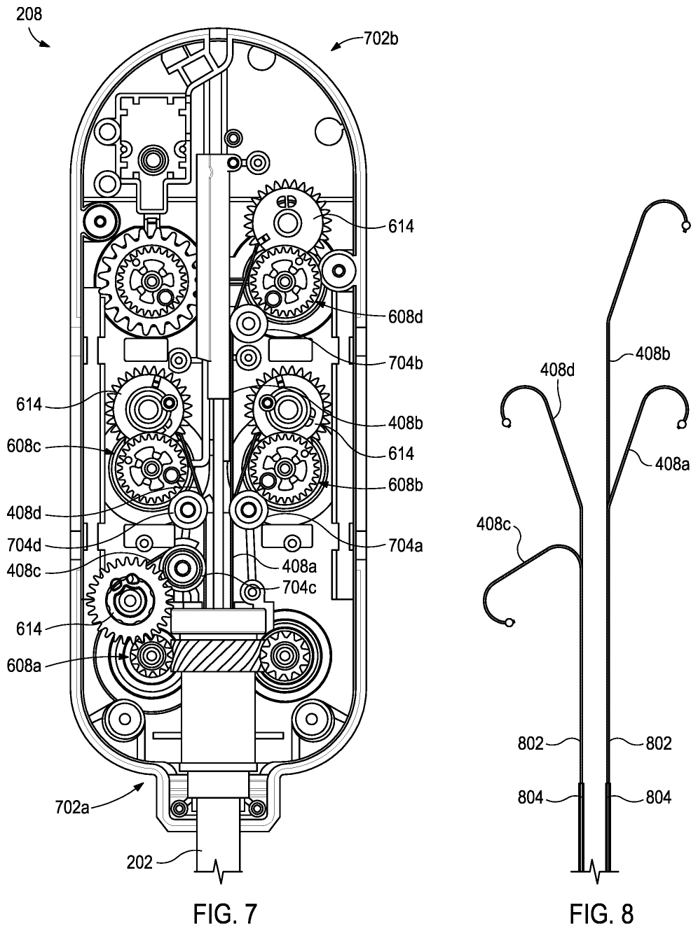

FIG. 7 is a top, exposed view of the drive housing 208, according to one or more embodiments. As illustrated, the drive housing 208 provides a first or “distal” end 702a through which the shaft 202 extends, and a second or “proximal” end 702b opposite the distal end 702a.

In some embodiments, one or more of the drive cables 408a-d may engage and otherwise wrap at least partially around an idler pulley rotatably mounted within the drive housing 208. Each idler pulley may re-direct the trajectory or cable pathway for the corresponding drive cable 408a-d before the drive cable 408a-d is coupled to the corresponding cable pulley 614 driven by the corresponding drive cable capstan 608a-d. In the illustrated embodiment, the first drive cable 408a engages and is re-directed by a first idler pulley 704a, the second drive cable 408b engages and is re-directed by a second idler pulley 704b, the third drive cable 408c engages and is re-directed by a third idler pulley 704c, and the fourth drive cable 408d engages and is re-directed by a fourth idler pulley 704d. In other embodiments, however, one or more of the idler pulleys 704a-d may be omitted, and the corresponding drive cable 408a-d may instead be received directly at the corresponding cable pulley 614.

As discussed above, the first drive cable capstan 608a is operatively coupled to or extends from the second input 506b (FIG. 5), the second drive cable capstan 608b is operatively coupled to or extends from the third input 506c (FIG. 5), the third drive cable capstan 608c is operatively coupled to or extends from the fourth input 506d (FIG. 5), and the fourth drive cable capstan 608d is operatively coupled to or extends from the fifth input 506e (FIG. 5). As depicted in FIG. 7, the second and third drive cable capstans 608b,c are laterally aligned within the drive housing 208 between the distal and proximal ends 702a,b and otherwise positioned at the same distance between the distal and proximal ends 702a,b. Accordingly, the second and third drive cable capstans 608b,c, and thus the third and fourth inputs 506c,d, may be characterized as “symmetric” or “symmetrically aligned”. In contrast, the first drive cable capstan 608a is arranged near the distal end 702a and the fourth drive cable capstan 608d is arranged near the proximal end 702b, such that the first and fourth drive cable capstans 608a,b are positioned at different distances between the distal and proximal ends 702a,b. Accordingly, the first and fourth drive cable capstans 608a,d, and thus the second and fifth inputs 506b,e, may be characterized as “asymmetric” or “asymmetrically aligned”.

Moreover, having the cable pulleys 614 arranged in different orientations (locations) with respect to the corresponding drive cable capstan 608a-d could also be characterized as an “asymmetric” relationship or “asymmetrically aligned”. For example, the third and fourth drive cable capstans 608c,b are symmetrically aligned between the distal and proximal ends 702a,b and on either side of the centerline of the drive housing 208, but the cable pulleys 614 associated with the third and fourth drive cable capstans 608c,b may not be symmetrically aligned. In such an example, one of the cable pulleys 614 may be arranged at a different angle between the pulley and capstan axes and would, therefore, be considered asymmetric. Those skilled in the art will readily appreciate that this may be done to create room for additional device functions, and could result in varying lengths of the drive cables 408a,d, and thus varying compliance.

Referring now to FIG. 8, with continued reference to FIG. 7, illustrated is a schematic view of the drive cables 408a-d as would be arranged within the drive housing 208 described herein. As illustrated, the first and fourth drive cables 408a,d, which are driven by actuation of the second and third drive cable capstans 608b,c, respectively, generally exhibit the same length or “cable pathway length”. In some embodiments, the first and fourth drive cables 408a,d may be actuated simultaneously to close the jaws 210, 212 (FIGS. 2 and 4) of the end effector 204 (FIGS. 2 and 4), which requires high load capacity. Accordingly, the first and fourth drive cables 408a,d may be characterized as “high load” cables. In contrast, the second and third drive cables 408b,c, which are driven by the fourth and first drive cable capstans 608d,a, respectively, exhibit different cable pathway lengths. In particular, the length of the second drive cable 408b is longer than the length of the third drive cable 408c. In some embodiments, the second and third drive cables 408b,c may be actuated simultaneously to open the jaws 210, 212, which requires low load capacity, and may thus be characterized as “low load” cables.

Depending on the design, length, and manufacture, each drive cable 408a-d may exhibit a unique stiffness, which equates to a unique or different compliance as driven in antagonistic relationships with other drive cables 408a-d using corresponding independent inputs 506b-e (FIG. 5). Different stiffnesses of the drive cables 408a-d require that the motors that drive the inputs 506b-e be operated at different speeds (torques) to comply with the stiffness of other drive cables 408a-d that may be operating antagonistically. This results in complicating the controls system to account for the varying compliances. Moreover, even with controls compensations for unequal stiffnesses, there will likely be decreased accuracy in wrist motion and jaw actuation.

According to embodiments of the present disclosure, the stiffness of a given drive cable 408a-d may be altered and otherwise optimized to match the stiffness of another drive cable 408a-d used in antagonistic operation, thus creating an equivalent compliance between the two motors used to drive the inputs 506b-e that actuate the two drive cables 408a-d. In such embodiments, the motors may be operated at the same speed, same torque, and same positional/rotational actuation, or any combination thereof. In other embodiments, the stiffness of all drive cables 408a-d may be altered and optimized to match each other. Matching the stiffness of the drive cables 408a-d may prove advantageous in simplifying the controls scheme. More specifically, drive cables 408a-d that exhibit the same stiffness allow the motors that drive the inputs 506b-e to actuate the drive cables 408a-d to be operated (rotated) at the same speed, the same torque, and the same position, or any combination thereof, since there is no need to compensate for different stiffnesses or compliance compensation. Moreover, altering or optimizing the stiffness of the drive cables 408a-d enables the asymmetric layout of the inputs 506b-e, thus providing more flexibility in the design for other device functionalities.

The stiffness of each drive cable 408a-d may be determined based on one or more characteristics of the drive cable 408a-d. More specifically, each drive cable 408a-d may include at least one flexible cable 802 and one or more stiffness components 804 coupled to the flexible cable 802. Characteristics of the flexible cable 802 and the stiffness component 804 for a given drive cable 408a-d may be altered to optimize the stiffness of the drive cable 408a-d.

The flexible cable 802 may be made of a flexible material capable of being wrapped around a pulley (e.g., the cable pulleys 614 of FIGS. 6A-6B). Example materials for the flexible cable 802 include, but are not limited to, a metal (e.g., tungsten, stainless steel, nitinol, etc.), a polymer (e.g., ultra-high molecular weight polyethylene), a synthetic fiber (e.g., KEVLAR®, VECTRAN®, etc.), an elastomer, or any combination thereof. Each of the foregoing materials exhibits a known stiffness, which may contribute to the overall stiffness of the drive cable 408a-d.

Another characteristic of the drive cable 408a-d that may determine its stiffness is the length or “cable pathway length” of the flexible cable 802. For example, the length of the flexible cable 802 for the first and fourth drive cables 408a,d is generally the same. If the material and the length of the flexible cable 802 are the same, the stiffness of cable 802 should also be the same. In contrast, the cable pathway length of the flexible cable 802 for the second and third drive cables 408b,c is different, thus resulting in a different stiffness for each cable 802. It should be noted, however, that even though the length is different, the stiffness of the cables 802 for the second and third drive cables 408b,c may be made similar or substantially similar by altering the material of each cable 802.

Another characteristic of the drive cable 408a-d that may determine its stiffness is the size (e.g., cross-sectional diameter) of the flexible cable 802. More specifically, a larger cross-sectional diameter of the flexible cable 802 will equate to a greater stiffness, whereas a smaller cross-sectional diameter of the flexible cable 802 will equate to a lesser stiffness. Moreover, whether the flexible cable 802 is braided or not, and what type of braiding construction is utilized, is another characteristic that may determine stiffness. In some embodiments, for example, the flexible cable 802 may comprise a braided cord made of any of the foregoing materials for the cable 802. In at least one embodiment, the braided cord may comprise braided stainless steel. A braided cable 802 may exhibit a higher stiffness as compared to a non-braided cable 802. Consequently, a braided cable 802 may exhibit a known stiffness that contributes to the overall stiffness of the drive cable 408a-d.

Other characteristics of the drive cable 408a-d that may be altered or optimized to determine its stiffness include the length, the material, and the size (cross-sectional diameter) of the stiffness component 804 coupled to the flexible cable 802. In some embodiments, the stiffness component 804 may comprise a long tube of a rigid material, such as a hypotube made of a metal (e.g., stainless steel) or a polymer, each of which exhibit a different stiffness. Accordingly, the stiffness of the drive cable 408a-d may be optimized by choosing a particular material for the stiffness component 804 and/or altering its length. Moreover, in some embodiments, there may be more than one stiffness component 804. In such embodiments, another characteristic of the drive cable 408a-d that may be altered or optimized to determine its stiffness may include how many stiffness components 804 are included in the drive cable 408a-d.

Accordingly, example characteristics of the drive cable 408a-d that may determine its stiffness include, but are not limited to, the length of the flexible cable 802, the material of the flexible cable 802, the cross-sectional diameter of the flexible cable 802, whether the cable is braided (and what type of braiding is used), the length of the stiffness component 804, the material of the stiffness component 804, the size (cross-sectional diameter) of the stiffness component 804, and the number of stiffness components 804. In some embodiments, one or more of the characteristics of the first and fourth drive cables 408a,d may be adjusted such that the stiffness of the first and fourth drive cables 408a,d is equalized. In other embodiments, one or more of the characteristics of the second and third drive cables 408b,c may be adjusted such that the stiffness of the second and third drive cables 408b,c is equalized. In yet other embodiments, one or more of the characteristics of each drive cable 408a-d may be adjusted such that the stiffness of each drive cable 408a-d is equalized.

In some embodiments, one or more of the drive cables 408a-d may be optimized such that its overall stiffness exhibits a predetermined differential or ratio between the stiffness of the flexible cable 802 and the stiffness of the stiffness component 804. For example, if the stiffness ratio (flexible to rigid) for a given drive cable 408a-d is near 1:1 it may be more difficult to tune compliance as compared to a drive cable 408a-d with a stiffness ratio (flexible to rigid) of 1:20. In some embodiments, the stiffness ratio between a stiffness of the stiffness component 804 and a stiffness of the flexible cable 802 is between 1:1 and 10:1. Moreover, in altering the characteristics to compensate for the overall stiffness of a given drive cable 408a-d, the one or more characteristics may be altered to increase or decrease the corresponding stiffness between 0% and 25%. Increasing or decreasing the corresponding stiffness between 0% and 25% constitutes a delta (change) shift from its original stiffness or stiffness rating.

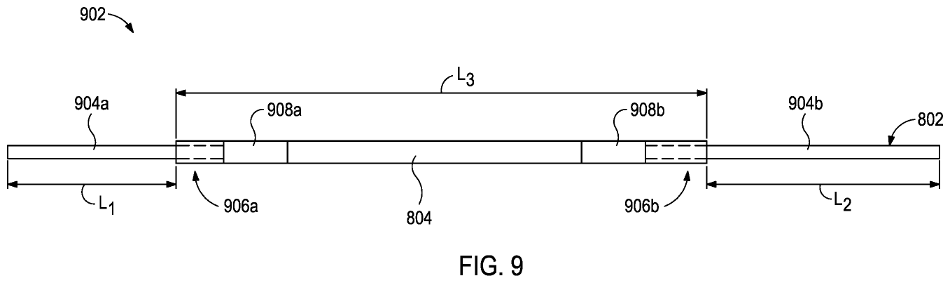

FIG. 9 is a schematic side view of an example drive cable 902, according to one or more embodiments. The drive cable 902 may be the same as or similar to any of the drive cables 408a-d discussed herein, and therefore may be best understood with reference thereto, where like numerals will represent like components not described again in detail. As illustrated, the drive cable 902 includes the flexible cable 802 and the stiffness component 804 coupled to the flexible cable 802. In the illustrated embodiment, however, the flexible cable 802 includes a distal section 904a coupled to a distal end 906a of the stiffness component 804, and a proximal section 904b coupled to a proximal end 906b of the stiffness component 804.

The distal section 904a is coupled to the stiffness component 804 at a first attachment 908a, and the proximal section 904b is coupled to the stiffness component 804 at a second attachment 908b. In some embodiments, one or both of the attachments 908a,b may comprise a crimped engagement, but could alternatively comprise other attachment means such as, but not limited to, an adhesive, a weld, brazing, an interface fit, one or more mechanical fasteners, a knot (braided attachment), or any combination thereof.

The distal section 904a extends distally from the stiffness component 804 and extends at least partially through the wrist 206 (FIGS. 2 and 4) to articulate the wrist 206 or operate the end effector 204 (FIGS. 2 and 4). The proximal section 904b extends proximally from the stiffness component 804 and to the drive housing 208 (FIGS. 6A-6B and 7) to be wrapped around a cable pulley 614 (FIGS. 6A-6B) and actuated, as generally described above. As illustrated, the distal section 904a may exhibit a first length L₁ and the proximal section 904b may exhibit a second length L₂. The first and second lengths L₁, L₂ may be the same or different. Moreover, the material and cross-sectional diameter of the distal and proximal sections 904a,b may be the same or different, depending on the application and stiffness requirements.

As illustrated, the stiffness component 804 exhibits a third length L₃, which may be adjusted as a compliance corrector that optimizes the overall stiffness of the drive cable 902. The material used for the stiffness component 804 may also help determine the overall stiffness of the drive cable 902.

Accordingly, example characteristics of the drive cable 902 that may determine its stiffness include, but are not limited to, the length L₁, L₂ of the distal and proximal sections 904a,b, the material of the distal and proximal sections 904a,b, the cross-sectional diameter (size) of the distal and proximal sections 904a,b, whether the distal and proximal sections 904a,b are braided, the length L₃ of the stiffness component 804, and the material of the stiffness component 804.

Embodiments disclosed herein include:

A. A surgical tool includes a drive housing having opposing distal and proximal ends, first and second drive cable capstans arranged within the drive housing and asymmetrically aligned between the distal and proximal ends, a first drive cable extending distally from the drive housing and actuatable by operation of the first drive cable capstan, and a second drive cable extending distally from the drive housing and actuatable by operation of the second drive cable capstan, wherein a stiffness of the first and second drive cables is equalized by adjusting one or more characteristics of one or both of the first and second drive cables.

B. A method of operating a surgical tool includes positioning the surgical tool adjacent a patient for operation, the surgical tool including a drive housing having opposing distal and proximal ends, first and second inputs rotatably mounted to a bottom of the drive housing, the first and second inputs being asymmetrically aligned between the distal and proximal ends, first and second drive cable capstans extending from the first and second inputs, respectively, a first drive cable extending distally from the drive housing and actuatable by operation of the first drive cable capstan, and a second drive cable extending distally from the drive housing and actuatable by operation of the second drive cable capstan, wherein a stiffness of the first and second drive cables is equalized by adjusting one or more characteristics of one or both of the first and second drive cables. The method further including adjusting one or more characteristics of one or both of the first and second drive cables and thereby equalizing a stiffness of the first and second drive cables, and actuating first and second motors to drive the first and second inputs, and thereby causing the first and second drive cables to move, wherein equalizing the stiffness of the first and second drive cables creates an equivalent compliance between the first and second motors.

Each of embodiments A and B may have one or more of the following additional elements in any combination: Element 1: further comprising a first input rotatably mounted to a bottom of the drive housing, the first drive cable capstan extending from the first input, and a second input rotatably mounted to the bottom of the drive housing, the second drive cable capstan extends from the second input, wherein the first and second inputs are asymmetrically aligned between the distal and proximal ends, and wherein equalizing the stiffness of the first and second drive cables creates an equivalent compliance between motors that drive the first and second inputs. Element 2: further comprising third and fourth cable capstans arranged within the drive housing and symmetrically aligned between the distal and proximal ends, a third drive cable extending distally from the drive housing and actuatable by operation of the third drive cable capstan, and a fourth drive cable extending distally from the drive housing and actuatable by operation of the fourth drive cable capstan, wherein a stiffness of the third and fourth drive cables is equal. Element 3: wherein the stiffness of the first and second drive cables is equalized to the stiffness of the third and fourth drive cables by adjusting one or more characteristics of one or more of the first, second, third, and fourth drive cables. Element 4: further comprising an elongate shaft that extends from the drive housing, and an end effector operatively coupled to a distal end of the elongate shaft and including opposing upper and lower jaws, wherein the first and second drive cables extend within the shaft toward the end effector and are actuatable to open the upper and lower jaws, and wherein the third and fourth drive cables extend within the shaft toward the end effector and are actuatable to close the upper and lower jaws. Element 5: wherein the end effector is selected from the group consisting of forceps, a tissue grasper, a needle driver, scissors, an electrosurgical tool, a stapler, a clip applier, and any combination thereof. Element 6: wherein each drive cable includes a flexible cable and a stiffness component coupled to the flexible cable, and wherein the one or more characteristics include a length of the flexible cable, a material of the flexible cable, a cross-sectional diameter of the flexible cable, whether the flexible cable is braided, a type of braiding construction of the flexible cable, a length of the stiffness component, a material of the stiffness component, a cross-section of the stiffness component, and whether there is more than one stiffness component. Element 7: wherein the flexible cable is made of a flexible material selected from the group consisting of a metal, a polymer, a synthetic fiber, an elastomer, and any combination thereof. Element 8: wherein a ratio of stiffness between a stiffness of the stiffness component and a stiffness of the flexible cable is between 1:1 and 10:1. Element 9: wherein each drive cable includes a flexible cable having a distal section and a proximal section, and a stiffness component having opposing distal and proximal ends, the distal section being coupled to the distal end of the stiffness component, and the proximal section being coupled to the proximal end of the stiffness component, and wherein the one or more characteristics include a length of the distal section, a length of the proximal section, a material of the distal and proximal sections, a cross-sectional diameter of the distal and proximal sections, whether one or both of the distal and proximal sections is braided, a type of braiding construction of one or both of the distal and proximal sections, a length of the stiffness component, a material of the stiffness component, a cross-section of the stiffness component, and whether there is more than one stiffness component. Element 10: wherein a ratio of stiffness between a stiffness of the stiffness component and a stiffness of the flexible cable is between 1:1 and 10:1. Element 11: wherein equalizing the stiffness of the first and second drive cables includes adjusting the one or more characteristics of one or both of the first and second drive cables and thereby altering the stiffness between 0% and 25%, wherein altering the stiffness between 0% and 25% constitutes a delta (change) shift from an original stiffness of the first and second drive cables. Element 12: further comprising first and second drive gears forming part of the first and second drive cable capstans, respectively, first and second driven gears positioned within the drive housing to be driven by the first and second drive gears, respectively as the first and second drive cable capstans rotate, and first and second cable pulleys coupled to the first and second driven gears, respectively, wherein the first drive cable is received by the first cable pulley, and the second drive cable is received by the second cable pulley.

Element 13: wherein adjusting the one or more characteristics comprises adjusting the one or more characteristics of one or both of the first and second drive cables and thereby altering the stiffness between 0% and 25%, wherein altering the stiffness between 0% and 25% constitutes a delta (change) shift from an original stiffness of the first and second drive cables. Element 14: wherein the surgical tool further includes third and fourth inputs rotatably mounted to the bottom of the drive housing and symmetrically aligned between the distal and proximal ends, third and fourth cable capstans extending from the third and fourth inputs, respectively, a third drive cable extending distally from the drive housing and actuatable by operation of the third drive cable capstan, and a fourth drive cable extending distally from the drive housing and actuatable by operation of the fourth drive cable capstan, wherein a stiffness of the third and fourth drive cables is equal. Element 15: further comprising equalizing the stiffness of the first and second drive cables to the stiffness of the third and fourth drive cables by adjusting one or more characteristics of one or more of the first, second, third, and fourth drive cables. Element 16: wherein the surgical tool further includes an elongate shaft that extends from the drive housing, and an end effector operatively coupled to a distal end of the elongate shaft and including opposing upper and lower jaws, the method further comprising actuating the first and second motors to move the first and second drive cables and thereby open the upper and lower jaws, and actuating third and fourth motors to drive the third and fourth inputs, and thereby causing the third and fourth drive cables to move and close the upper and lower jaws. Element 17: wherein each drive cable includes a flexible cable and a stiffness component coupled to the flexible cable, and wherein adjusting the one or more characteristics of one or both of the first and second drive cables includes at least one of the following changing a material of the flexible cable for one or both of the first and second drive cables, and adjusting a length of the stiffness component for one or both of the first and second drive cables. Element 18: wherein each drive cable includes a flexible cable and a stiffness component coupled to the flexible cable, and wherein adjusting the one or more characteristics of one or both of the first and second drive cables includes adjusting one or more of the following a length of the flexible cable, a material of the flexible cable, a cross-sectional diameter of the flexible cable, whether the flexible cable is braided, a length of the stiffness component, a material of the stiffness component, a cross-section of the stiffness component, and whether there is more than one stiffness component.

By way of non-limiting example, exemplary combinations applicable to A and B include: Element 2 with Element 3; Element 2 with Element 4; Element 4 with Element 5; Element 6 with Element 7; Element 6 with Element 8; and Element 9 with Element 10.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

What is claimed is:

1. A surgical tool, comprising:
a drive housing having opposing distal and proximal ends;
first and second drive cable capstans arranged within the drive housing and asymmetrically aligned between the distal and proximal ends;
a first drive cable extending distally from the drive housing and actuatable by operation of the first drive cable capstan; and
a second drive cable extending distally from the drive housing and actuatable by operation of the second drive cable capstan,
wherein a stiffness of the first and second drive cables is equalized by adjusting one or more characteristics of one or both of the first and second drive cables.

2. The surgical tool of claim 1, further comprising:
a first input rotatably mounted to a bottom of the drive housing, the first drive cable capstan extending from the first input; and
a second input rotatably mounted to the bottom of the drive housing, the second drive cable capstan extends from the second input,
wherein the first and second inputs are asymmetrically aligned between the distal and proximal ends, and
wherein equalizing the stiffness of the first and second drive cables creates an equivalent compliance between motors that drive the first and second inputs.

3. The surgical tool of claim 1, further comprising:
third and fourth cable capstans arranged within the drive housing and symmetrically aligned between the distal and proximal ends;
a third drive cable extending distally from the drive housing and actuatable by operation of the third drive cable capstan; and
a fourth drive cable extending distally from the drive housing and actuatable by operation of the fourth drive cable capstan,
wherein a stiffness of the third and fourth drive cables is equal.

4. The surgical tool of claim 3, wherein the stiffness of the first and second drive cables is equalized to the stiffness of the third and fourth drive cables by adjusting one or more characteristics of one or more of the first, second, third, and fourth drive cables.

5. The surgical tool of claim 3, further comprising:
an elongate shaft that extends from the drive housing; and
an end effector operatively coupled to a distal end of the elongate shaft and including opposing upper and lower jaws,
wherein the first and second drive cables extend within the shaft toward the end effector and are actuatable to open the upper and lower jaws, and
wherein the third and fourth drive cables extend within the shaft toward the end effector and are actuatable to close the upper and lower jaws.

6. The surgical tool of claim 5, wherein the end effector is selected from the group consisting of forceps, a tissue grasper, a needle driver, scissors, an electrosurgical tool, a stapler, a clip applier, and any combination thereof.

7. The surgical tool of claim 1, wherein each drive cable includes a flexible cable and a stiffness component coupled to the flexible cable, and wherein the one or more characteristics include:
a length of the flexible cable;
a material of the flexible cable;
a cross-sectional diameter of the flexible cable;
whether the flexible cable is braided;
a type of braiding construction of the flexible cable;
a length of the stiffness component;
a material of the stiffness component;
a cross-section of the stiffness component; and
whether there is more than one stiffness component.

8. The surgical tool of claim 7, wherein the flexible cable is made of a flexible material selected from the group consisting of a metal, a polymer, a synthetic fiber, an elastomer, and any combination thereof.

9. The surgical tool of claim 7, wherein a ratio of stiffness between a stiffness of the stiffness component and a stiffness of the flexible cable is between 1:1 and 10:1.

10. The surgical tool of claim 1, wherein each drive cable includes:
a flexible cable having a distal section and a proximal section; and
a stiffness component having opposing distal and proximal ends, the distal section being coupled to the distal end of the stiffness component, and the proximal section being coupled to the proximal end of the stiffness component, and
wherein the one or more characteristics include:
a length of the distal section;
a length of the proximal section;
a material of the distal and proximal sections;
a cross-sectional diameter of the distal and proximal sections;
whether one or both of the distal and proximal sections is braided;
a type of braiding construction of one or both of the distal and proximal sections;
a length of the stiffness component;
a material of the stiffness component;
a cross-section of the stiffness component; and
whether there is more than one stiffness component.

11. The surgical tool of claim 10, wherein a ratio of stiffness between a stiffness of the stiffness component and a stiffness of the flexible cable is between 1:1 and 10:1.

12. The surgical tool of 1, wherein equalizing the stiffness of the first and second drive cables includes adjusting the one or more characteristics of one or both of the first and second drive cables and thereby altering the stiffness between 0% and 25%, wherein altering the stiffness between 0% and 25% constitutes a delta (change) shift from an original stiffness of the first and second drive cables.

13. The surgical tool of claim 1, further comprising:
first and second drive gears forming part of the first and second drive cable capstans, respectively;
first and second driven gears positioned within the drive housing to be driven by the first and second drive gears, respectively as the first and second drive cable capstans rotate; and
first and second cable pulleys coupled to the first and second driven gears, respectively,
wherein the first drive cable is received by the first cable pulley, and the second drive cable is received by the second cable pulley.

14. A method of operating a surgical tool, comprising:
positioning the surgical tool adjacent a patient for operation, the surgical tool including:
a drive housing having opposing distal and proximal ends;
first and second inputs rotatably mounted to a bottom of the drive housing, the first and second inputs being asymmetrically aligned between the distal and proximal ends;
first and second drive cable capstans extending from the first and second inputs, respectively;
a first drive cable extending distally from the drive housing and actuatable by operation of the first drive cable capstan; and
a second drive cable extending distally from the drive housing and actuatable by operation of the second drive cable capstan, wherein a stiffness of the first and second drive cables is equalized by adjusting one or more characteristics of one or both of the first and second drive cables.
adjusting one or more characteristics of one or both of the first and second drive cables and thereby equalizing a stiffness of the first and second drive cables; and
actuating first and second motors to drive the first and second inputs, and thereby causing the first and second drive cables to move, wherein equalizing the stiffness of the first and second drive cables creates an equivalent compliance between the first and second motors.

15. The method of claim 14, wherein adjusting the one or more characteristics comprises adjusting the one or more characteristics of one or both of the first and second drive cables and thereby altering the stiffness between 0% and 25%, wherein altering the stiffness between 0% and 25% constitutes a delta (change) shift from an original stiffness of the first and second drive cables.

16. The method of claim 14, wherein the surgical tool further includes:
third and fourth inputs rotatably mounted to the bottom of the drive housing and symmetrically aligned between the distal and proximal ends;
third and fourth cable capstans extending from the third and fourth inputs, respectively;
a third drive cable extending distally from the drive housing and actuatable by operation of the third drive cable capstan; and
a fourth drive cable extending distally from the drive housing and actuatable by operation of the fourth drive cable capstan, wherein a stiffness of the third and fourth drive cables is equal.

17. The method of claim 14, further comprising equalizing the stiffness of the first and second drive cables to the stiffness of the third and fourth drive cables by adjusting one or more characteristics of one or more of the first, second, third, and fourth drive cables.

18. The method of claim 14, wherein the surgical tool further includes an elongate shaft that extends from the drive housing, and an end effector operatively coupled to a distal end of the elongate shaft and including opposing upper and lower jaws, the method further comprising:
actuating the first and second motors to move the first and second drive cables and thereby open the upper and lower jaws; and
actuating third and fourth motors to drive the third and fourth inputs, and thereby causing the third and fourth drive cables to move and close the upper and lower jaws.

19. The method of claim 14, wherein each drive cable includes a flexible cable and a stiffness component coupled to the flexible cable, and wherein adjusting the one or more characteristics of one or both of the first and second drive cables includes at least one of the following:
changing a material of the flexible cable for one or both of the first and second drive cables; and
adjusting a length of the stiffness component for one or both of the first and second drive cables.

20. The method of claim 14, wherein each drive cable includes a flexible cable and a stiffness component coupled to the flexible cable, and wherein adjusting the one or more characteristics of one or both of the first and second drive cables includes adjusting one or more of the following:
a length of the flexible cable;
a material of the flexible cable;
a cross-sectional diameter of the flexible cable;
whether the flexible cable is braided;
a length of the stiffness component;
a material of the stiffness component;
a cross-section of the stiffness component; and
whether there is more than one stiffness component.
